(19) United States
(12) Patent Application Publication
(10) Pub. No.: US 2015/0174208 A1
(43) Pub. Date: Jun. 25, 2015

(54) Cosmetic and pharmaceutical composition with modified olygopeptides in form of supramolecular assembly

(71) Applicants: Artur Martynov, Kharkov (UA); Boris S. Farber, Brooklyn, NY (US); Sonya Sophya Farber, New York, NY (US)

(72) Inventors: Artur Martynov, Kharkov (UA); Boris S. Farber, Brooklyn, NY (US); Sonya Sophya Farber, New York, NY (US)

(21) Appl. No.: 14/140,489

(22) Filed: Dec. 25, 2013

(51) Int. Cl. A61K 38/28; A61Q 19/00; A61K 8/66; A61K 8/64; A61K 38/47; A61K 38/48

(52) U.S. Cl. CPC A61K 38/28; A61K 38/47; A61K 38/4886; A61K 8/66; A61K 8/64; A61Q 19/00

(57) ABSTRACT

The invention is a cosmetic and pharmaceutical liposomal composition which comprises a mixture of carboxylated oligopeptides derived from natural polypeptides; insulin; hyaluronidase and collagenase by their enzymatic hydrolysis; followed by a carboxylation of the free amino groups of lysine, histidine, and additional hydrolysis of terminal amino groups in the oligopeptides. Natural polypeptides may be hydrolyzed with a proteolytic enzyme to form a mixture of oligopeptides and succinylated them. Next, the resulting mixture was injected into the liposomes by classical methods to improve the intestinal absorption and bioavailability in the body, prolonging the action of the system. Such a system behaves as quasi-live and is capable of forming supramolecular complexes with the original proteins, such as insulin and acylated insulin oligopeptides, or insulin receptors on a cell and acylated insulin's oligopeptides. These cosmetic formulations may be used as prophylactic and therapeutic drugs in the prevention of infectious diseases as do vaccines in the treatment of cancer; pancreatitis and other diseases; rejuvenation of skin and body; and acceleration of wound healing.

### CROSS REFERENCE TO RELATED APPLICATIONS

The present application is a continuation-in-part of the application: Ser. No. 12/931,463, filed Feb. 1, 2011, which is a continuation of the International Application No. PCT/RU2010/000697, filed Nov. 22, 2010.

The present application is a continuation-in-part of the application: Ser. No. 14/023,231, filed Sep. 10, 2013, which is a continuation-in-part of the applications:
Ser. No. 12/931,467, filed Feb. 1, 2011, which is a continuation of the International Application No. PCT/RU2010/000701, filed Nov. 22, 2010,
Ser. No. 12/931,466, filed Feb. 1, 2011, which is a continuation of the International Application No. PCT/RU2010/000700, filed Nov. 22, 2010,
Ser. No. 12/931,461, filed Feb. 1, 2011, which is a continuation of the International Application No. PCT/RU2010/000692, filed Nov. 22, 2010, and
Ser. No. 12/931,458, filed Feb. 1, 2011, which is a continuation of the International Application No. PCT/RU2010/000690, filed Nov. 22, 2010.

### TECHNICAL FIELD

This invention relates to medicine and pharmaceuticals, specifically, to pharmaceutical compositions and methods of manufacturing pharmaceutical compositions.

### TERMINOLOGY

The term “Cosmetic and pharmaceutical composition” represents a combination of carboxylated oligopeptides obtained by hydrolysis of protein: insulin, collagenase, hyaluronidase, partially acylating carboxylated amino acid residues, and terminal basic amino acids in the structure of the oligopeptides. This composition is then introduced into the liposome by classical methods. Also, the composition may include a pharmaceutical acceptable auxiliaries such as glycerol, PEG-400, preservatives, stabilizers, thermal- and cryo-protectors.

Application of only one carboxylated oligopeptide from this mixture as a pharmaceutical or cosmetic product is not recommended, since only a mixture of various oligopeptides is capable of forming with one other as well as with the target insoluble stable supramolecular assemblies.

The term “enzymatic hydrolysis” refers to a protease enzyme that is catalyzed or synthesized by analogs of proteins to protease degradation of smaller oligomer fragments of oligopeptides. The size of oligopeptides in this hydrolysis ranges from 2 amino acids up to 30 amino acid residues.

“Natural polypeptides” refers to insulin, collagenase, or hyaluronidase in either a mixture or by itself.

“Carboxylation” refers to administering the oligopeptides to the structure of the carboxyl groups through the formation of a new covalent bond method using an acylation with anhydrides of dicarboxylic acids. When administering polycarboxylic acid anhydrides, an amide group is formed from the amino group remainder of lysine, histidine, and terminal amino acid.

The term “partially acylating” involves only substituting parts of amino groups with the basic amino acid residues (lysine, histidine, terminal amino group) to the oligopeptide. The remaining parts of lysine, histidine, and the amino end groups will not be substituted. The degree of substitution was calculated either from the formulas of combinatorial mathematics to obtain the maximum number of combinations and substitutions, or empirically through the synthesis of a plurality of derivatives with varying degrees of modification and selection of the most biologically active compositions.

The term “amino acid residues having free amino groups” refers to residues of lysine, histidine, and the amino end groups forming during the hydrolysis of proteins with proteolytic enzymes.

### SUMMARY OF INVENTION

The invention is a cosmetic and pharmaceutical liposomal composition which comprises a mixture of carboxylated oligopeptides; polypeptides derived from the natural insulin; hyaluronidase and collagenase by their enzymatic hydrolysis; followed by a carboxylation of the free amino groups of lysine, histidine, and additional hydrolysis of terminal amino groups in the oligopeptides. Natural polypeptides may be hydrolyzed with a proteolytic enzyme to form a mixture of oligopeptides. The resulting mixture may be subjected to the carboxylation of oligopeptides: substituted amino groups of lysine residues; histidine; and released amino groups as a result of hydrolysis by acylation with anhydrides of polycarboxylic acids such as succinic acids. The ratio modifier and mixtures of oligopeptides are calculated using the formulas of combinatorial mathematics in order to maximize the diversity of derivatives in one volume (Example 1). Next, the resulting mixture is injected into the liposomes by classical methods in order to improve the intestinal absorption and bioavailability in the body, thus prolonging the action of the system. Such a system behaves as quasi-live and is capable of forming supramolecular complexes with original proteins such as insulin; acylated insulin oligopeptides; and insulin receptor of a cell. Often such complex supramolecular complexes are several hundred times more active than the parent protein.

Such a system is capable of following a single oral administration to maintain the normal blood glucose in diabetic animals. Auxiliary substances, such as preservatives, stabilizers, plasticizers, and others may also be included in the composition. The proposed composition may be used in the form of a sterile injectable, including the infusion form of tablets, capsules, suppositories, solution, syrups, ointments, creams, patches, and other forms that are commonly used in the pharmaceutical and cosmetic industries. These cosmetic formulations may be used as prophylactic and therapeutic drugs in the prevention of infectious diseases as vaccines in the treatment of oncological diseases, pancreatitis, other diseases, skin and body rejuvenation, as well as accelerate wound healing.

### DETAILED DESCRIPTION OF THE INVENTION, EXAMPLES

### Example 1

### Quasi-Living Insulin

Diabetes mellitus is one of the most common serious diseases that is based on either an absolute or relative lack of the pancreas hormone—insulin. Insulin therapy (insulin administration from the outside) is a traditional and single method of treating the disease by allowing to compensate for the lack of insulin in the body.

The most common method of insulin administration is by a subcutaneous injection. This method is inconvenient; traumatizing for patients (especially children); causes physical and emotional suffering; but most importantly, it may exacerbate the pathology of the disease. The latter is due to the fact that with subcutaneous injection of insulin, normal blood glucose levels are achieved through systematic hyperinsulinemia in peripheral tissues, whereas the liver (the main place of activity of the endogenous insulin produced in the body), is lacking insulin.

The only way to prevent the complications inevitably associated with insulin injection, is by achieving, whenever possible, a complete simulation of the natural pathways of hormone supply in a living organism—i.e., to simulate the physiological difference in the insulin levels in the portal and peripheral circulatory systems. From this point of view, the oral (by mouth) way of insulin delivery is the most favorable.

The main obstacles hindering the creation of the oral forms of insulin are the hormone's low resistance to the action of proteolytic enzymes in the gastrointestinal tract and low permeability of insulin through the epithelial tissue of the intestinal wall into the bloodstream that is due to low lipophilicity as well as the large size of hormone macromolecules.

Over the past decades there have been numerous attempts to create oral forms of insulin, however, an effective drug that could compete with intravenously injected insulin on the therapeutic action has not yet been created. Among the pharmaceutical forms of oral medications, the most attractive and promising is the solid form, since it is the most comfortable and convenient in application, as well as in storage. In addition, the production technologies of these forms are relatively inexpensive and sufficiently developed.

We have proposed the new quasi-living self-organizing system for the purpose of creating pharmaceutical oral forms of insulin. The system is a mixture of insulin oligopeptides with artificially increased negative charges of the molecules. The first stage of modification is the enzymatic hydrolysis of insulin molecule (in this case proteinase K). Next, the structure of the synthesized oligopeptides is partially modified in order to replace a part of positive charges in amino groups of lysine and histidine for the carboxyl residues of dicarboxylic acids. Partial modification is actually a combinatorial synthesis that leads to the formation of thousands of different peptides with different structure and specificity. Such a system is protected from the action of intestinal proteolytic enzymes, as it has been already hydrolized, and consists of small oligopeptides. It is freely absorbed from the intestine due to the small size of its molecules and like a complement, it can be collected on the insulin receptors into insulin-protein assembly.

Self-assembly of supramolecular peptide systems is also well-studied in bacteriophages. Initially, the number of modified peptides is redundant to ensure the process of self-organization in the insulin receptor. If the body of a diabetic has insulin antibodies or if receptors do not match the insulin structure (tolerance to insulin in type 2 diabetics); also, if the number of insulin receptors is insufficient the quasi-living system is capable of self-organization and self-assembly. It automatically picks up from excessive peptides only those components of the “mosaic” that lead to the establishment of a truly effective quasi-insulin on the receptor. Antibodies do not affect these peptides, since the structure of peptides differs from that of insulin. Small size of the composite oligopeptides and excess negative charge of molecules block generation of antibodies and contribute to the long-term effect of the drug and provide the opportunity to apply such systems orally. Previously, α- and γ-interferons have been also modified by us with the application of quasi-living systems' technology and they have shown completely new properties.

The purpose of the research was to obtain an oral form of insulin on the basis of quasi-living self-assembled and self-organized system of acylated peptides derived from enzymatic hydrolyzate of insulin (MI), and to study the effectiveness of the resulting system on the model of alloxan diabetes in rats.

### Example 2

### Synthesis of MI on the Basis of Insulin's Succinylated Peptides

Crystalline insulin (Indar, Ukraine) in the amount of 100 mg was dissolved in 1 ml of 0.1 M hydrochloric acid and then enzymatically hydrolyzed by incubation with proteinase K (Fluka, 400 ED/mg) at room temperature for 1 hour. Then, while stirring the solution, the powdered succinic anhydride (7.5 mg) was added slowly and incubated with stirring for 60 minutes. The resulting peptides were purified of salts in column Sephadex G-25, with TRIS-hydrochloride as the eluent. The yield of protein was controlled by the absorption of the eluate in the UV region of the spectrum, at 280 nm. Salt-free peptides were poured into vials and lyophilized. Further the hypoglycemic effect of MI on the model of alloxan diabetes was studied in rats: at rest and during glucose load. Input control of insulin was provided using the microfluidic method at bioanalyzer Agilent-2100, chip Protein-80. MI was analyzed using high pressure liquid chromatograph at Millichrom-A-02 (Novosibirsk, Russian Federation) in the Microcolumn, Hypersil-18 at a pressure of 30 kPa 5% ACN, 50 mM ADHP to 60% ACN, 50 mM ADPH. MI was dissolved in 0.9% sodium chloride. Similarly, we can get the acylated enzyme collagenase and hyaluronidase hydrolysates.

### Example 3

### Receiving Liposomal Oligopeptides' Forms of Succinylated Insulin Orally

Obtained acylated insulin's hydrolyzate oligopeptides dissolved in 0.9% sodium chloride solution to form a solution equivalent to 4.3 mg of protein/ml as preservatives such as sodium ascorbate, benzalkonium chloride, methyl and propyl parabens can be used as well as to enhance the absorption of peptides-up to 3% of the PEO-400. The solution was injected into the suspension of phosphatidylcholine lyophilized liposomes (drug Lipine, Biolek, Kharkov). In order to prepare the liposomes, a mixture of phosphatidylcholine and phosphatidylethanolamine with cholesterol was mixed. The resulting mixture was treated with a 3 minute sonication at 44 kHz and at a temperature not higher than +50° C. Liposomes with oligopeptides can also be obtained by any other standard methods such as freeze-reversed phases. The resulting solution was a 5-fold (relative to the injectable form) dose that was orally administered to animals.

The study of hypoglycemic action of MI administered orally to a model of alloxan diabetes

The experiments used 80 white (albino) male rats of Vistar, weighing 180-220 g. The care of the animals was provided in standard vivarium conditions. They were maintained in standard environmental conditions of temperature (22-25° C.), relative humidity (60 -70%), dark/light cycle, and fed a standard diet and water ad libitum. All animal procedures were performed according to the Guide for the Care and Use of Laboratory Animals of the National Institutes of Health, as well as according to guidelines set by the Animal Welfare Act. Diabetes was induced by single intraperitoneal injections of alloxan monohydrate at a dose of 120 mg/kg, freshly prepared from 0.9% sodium chloride solution. The animals were deprived of food for 24 hours before the injection of alloxan.

Diabetes was fully developed in rats 72 hours after the injection of the toxin, as evidenced by the level of glucose in the blood serum. For this study, rats were selected with a fasting glucose content above 11.1 mmol/l (fasting blood glucose.) Glucose content in blood, taken from the tail vein was determined using glucometer “One touch Ultra” (USA).

In the first set of experiments, without glucose, the animals were distributed into 4 groups of 10 animals per group: 1—intact control (saline administration); 2—intact rats injected with the modified insulin (MI); 3—diabetes control (infusion of saline solution); 4—animals with diabetes injected with MI.

Rats were fed for 18 hours before and 3 hours after administration of insulin and placebo. Modified insulin was administered in a dose of 50 U/kg, that is 5 times higher than the dose effective in rats (10 U/kg) according to the references. The drug was dissolved in saline at the rate of 25 IU/ml and was administered through an intragastric probe in a dose of 0.2 ml/100 g. The control animals were injected a saline solution in similar doses. Glucose content in rats blood was assessed prior to drug administration and then in 0.5, 1, 2, 3 and 24 hours thereafter.

In the second experimental setup, with a load of glucose, animals were distributed into 2 groups of 10 animals per group: 1—diabetes control (infusion of saline solution); 2—diabetic animals injected with MI.

The animals were deprived of food for 18 hours before the start of the experiment. Feeding was provided after taking blood samples for the three hour experiment. MI was given per os, in a dose of 50 U/kg, animal control group received saline. After 15 minutes, rats were injected with glucose in a dose of 3 g/kg (40% solution, 0.75 mg/100 g). In the experiments, the drug Glucose was used—the 40% injection solution in vials of 20 ml, manufactured by JSC “Farmak” (Kiev, Ukraine). Immediately before MI injection and 0.5, 1, 2, 3 and 24 hours after the load of the glucose content in the blood serum was determined.

The research results are processed with the method of variational statistics using Student's test, with a significance level of P≦0.05. The data are presented in Table 1.

Partial acylation of these peptides is calculated according to the laws of combinatorics to obtain the maximum number of peptide derivatives. The ratio of insulin moles that should be modified to moles of anhydride is calculated according to combinatorial equation:

m=(2ⁿ−1), (1)

where:

m—number of molecules (and moles) of insulin, which must be modified to obtain the maximum amount of various insulin derivatives, this value for insulin is equal to 131,071

n—number of amino acid residues available for modification by anhydride in one insulin molecule (it is conditionally accepted that insulin is not hydrolyzed, and represents the whole molecule)

$$k = \frac{n ( {2}^{n} - 1 ) + n}{2} = n {2}^{( n - 1 )} \qquad ( 2 )$$

where:

k—number of moles of succinic anhydride, which is necessary for the modification of a protein molecule containing n groups available for modification.

In our case, n=17, k=1114112. Thus, for the modification of 131,071 mol of insulin, 1,114,112 mol of succinic anhydride are required. This results in 131,071 different molecules of succinylated insulin. The molar ratio of anhydride to insulin is 8.5:1. In this case, the synthesis will be observed of the maximum number of different insulin derivatives capable of interaction and self-organization into the supramolecular structure of quasi-insulin on the insulin receptor.

TABLE 1
Dynamics of glucose content in blood of rats with alloxan diabetes after
single oral administration of insulin peptide supramolecular assembly

| Groups of | Glucose content in blood serum, mmol/l | | | | | |
|---|---|---|---|---|---|---|
| animals | Initial level | 0.5 h | 1 h | 2 h | 3 h | 24 h |
| Control | 4.68 ± 0.20 | 4.76 ± 0.23 | 4.61 ± 0.19 | 4.62 ± 0.15 | 4.51 ± 0.20 | 4.71 ± 0.18 |
| Control + MI | 4.57 ± 0.18 | 4.58 ± 0.19 | 4.67 ± 0.18 | 4.71 ± 0.13 | 4.67 ± 0.13 | 4.56 ± 0.15 |
| Diabetes | 19.48 ± 1.77 | 19.83 ± 1.53 | 18.24 ± 1.34 | 17.58 ± 1.36 | 16.23 ± 1.43 | 19.49 ± 1.29 |
| Diabeted + | 18.82 ± 1.00 | 15.50 ± 1.2² | 11.99 ± 1.22^{1,2} | 9.24 ± 1.34^{1,2} | 7.74 ± 1.56^{1,2} | 11.28 ± 1.39^{1,2} |
| MI | | | | | | |

Notes:
¹Statistically significant differences relative to baseline values
²Statistically significant differences between groups of Diabetes and Diabetes + MI (p < 0.05)

The modified insulin hypoglycemic action was studied in experiments conducted on rats with alloxan—induced type I diabetes. The results were compared with action of placebo and with intact control. As follows from the data in Table 1, the introduction of the modified insulin into intact animals (without diabetes) did not result in statistically significant changes in blood glucose levels. At the same time the introduction of the modified insulin to diabetic animals caused a significant change in this indicator. In the diabetes control group, a gradual slight decrease in blood glucose was observed associated with the lack of food in animals. It is known that in diabetes blood glucose is not a stable and tightly controlled parameter, as it is the case in healthy animals. Within 30 minutes after MI introduction, a decrease of glycemia was detected. In all subsequent periods the glucose level decreased, and the differences were significant, both in relation to the original data, and to diabetes control. Three hours later the figures reached almost normal levels and were more than 2 times lower than in the diabetes control group. The rates were significantly lower and 24 hours after MI administration.

The important aspects of MI action are:

1) Gradual pattern of changes, which exclude the formation of diabetic hypoglycemia observed with the introduction of injectable forms of insulin. If our hypothesis is correct, this can be explained by the length of the process of insulin molecule self-assembly. This may also explain the lack of glucose reduction in intact animals treated by MI. The duration of the process allows activation of compensatory mechanisms that support a stable glucose level in healthy organism (glucagon production, etc.)

2) Exceptional duration of the effect—is up to 24 hours. The phenomenon can be explained by the following considerations. First, the structure of modified peptides of insulin may differ from the structure of native insulin. This makes them inaccessible to the action of the first enzyme that metabolizes insulin-hepatic glutathione-insulin transhydrogenase that is characterized by a high substrate specificity. Secondly, the modified peptides of insulin, as noted above, may be unresponsive to insulin antibodies—their production does not occur, leaving MI active for a long time.

The action of MI is most clearly manifested under a standard glucose load in the background of fasting for 18 hours. The data show that introduction of MI drastically alters the glycemic curves characteristic of diabetes. There is no distinct increase in glucose level within the first 1-2 hours after the load. The curve in this period is smoothed, and within 3 hours the glucose level is reduced to almost normal values. As in the previous experimental setup, 24 hours after administration of the MI blood glucose was also significantly lower than in the diabetes control. Consequently, MI not only reduces glycemia smoothly within 24 hours, but also “takes care” of its postprandial increase.

Thus, MI has the following advantages:

1. mild action, absence of evident hypoglycemia

2. prolonged effect

3. smoothing of postprandial hyperglycemia

Of course, the present paper provides only a limited explanation of the obtained phenomena, and further research and testing is required. It is also necessary to evaluate the possibility of long-term introduction of MI, and to identify the effective dose for this mode of introduction (it can possibly be reduced.)

### CONCLUSIONS

The quasi-living system based on combinatorial acylated derivatives of hydrolyzed insulin has shown high biological activity when administered orally in rats with alloxan diabetes. The system promoted reduction in glucose level to 10 mmol/L on average, and maintained this level within 24 hours after a single application. It can be considered a candidate for development and implementation in the capacity of oral insulin. Efficiency of the preparation was confirmed in animals by using both fasting and glucose load.

1. A cosmetic or pharmaceutical composition comprising liposomes with a mixture of succinylated oligopeptides in the form of a supramolecular assembly, wherein the succinylated oligopeptides are obtained by enzymatic hydrolysis of natural polypeptides, wherein the natural polypeptides are selected from a mixture of collagenase, hyaluronidase and insulin, which results in an oligopeptide mixture ranging from 2 to 30 amino acid residues; afterwards succinylation of amino acid residues having a free amino groups in the oligopeptide mixture is done by partially acylating the mixture, the oligopeptides are acylated at a modification level of 0.1 to 10% of their mass.

2. (canceled)

3. The composition according to claim 1, wherein the liposomes are obtained by a reverse phase method, followed by an ultrasound processing of the liposomes.

4. The composition according to claim 1, wherein the liposomes are obtained by the freezing-thawing method.

5. The composition according to claim 1, wherein the liposomes are obtained by a method of introducing the protein solution to a suspension of lyophilized liposomes.

6. The composition according to claim 1, wherein the liposomes are derived from phosphatidylcholine.

7. The composition according to claim 1, wherein the liposomes are derived from phosphatidylethanolamine.

8. The composition according to claim 1, wherein the liposomes are derived from a mixture of phosphatidylcholine and phosphatidylethanolamine.

9. A method of obtaining a cosmetic and pharmaceutical composition comprising liposomes with a mixture of carboxylated oligopeptides, obtained by enzymatic hydrolysis of natural polypeptides, that resulted in a oligopeptide mixture ranging of 2 to 30 amino acid residues, and carboxylation of amino acid residues having a free amino groups in the oligopeptide mixture by acylating with succinic anhydride or alkylating with monochloroacetic acid, wherein the oligopeptides are acylated at modification level of 0.1 to 10% of their mass and are present in the form of a supramolecular assembly, wherein the proteins are derived from an at least one of collagenase, hyaluronidase, and insulin.

10. The method according to claim 13, wherein the partially acylated protein is hyaluronidase.

11. The method according to claim 13, wherein the partially acylated protein is collagenase.

12. The method according to claim 13, wherein the partially acylated protein is insulin.

13. The method according to claim 13, wherein the partially acylated protein is a mixture of collagenase, hyaluronidase, and insulin.

14. The method according to claim 13 wherein the partially acylated proteins are produced via a reaction with succinic anhydride.

15. The method according to claim 13, wherein the liposomes are obtained by a reverse phase method, followed by an ultrasound treatment of the liposomes.

16. The method according to claim 13, wherein the liposomes are obtained by freezing-thawing.

17. The method according to claim 13, wherein the liposomes are obtained by introducing a protein solution into a suspension of lyophilized liposomes.

18. The method according to claim 13, wherein the liposomes are derived from phosphatidylcholine.

19. The method according to claim 13, wherein the liposomes are derived from phosphatidylethanolamine.

20. The method according to claim 13, wherein the liposomes are derived from a mixture of phosphatidylcholine and phosphatidylethanolamine.
